(19) United States
(12) Patent Application Publication
(10) Pub. No.: US 2016/0074749 A1
(43) Pub. Date: Mar. 17, 2016

(54) METHOD AND APPARATUS FOR PROVIDING PREFRONTAL ACTIVITY GAME

(71) Applicant: KOREA UNIVERSITY RESEARCH AND BUSINESS FOUNDATION, Seoul (KR)

(72) Inventor: Kyuwan Choi, Goyang-si (KR)

(21) Appl. No.: 14/541,643

(22) Filed: Nov. 14, 2014

(30) Foreign Application Priority Data
Sep. 12, 2014 (KR) 10-2014-0120849

(51) Int. Cl. A63F 13/213; A63F 13/537; A61B 5/0496

(52) U.S. Cl. CPC A63F 13/213; A61B 5/0496; A63F 13/537

(57) ABSTRACT

A method for providing a prefrontal activity game is provided. The method includes providing a visual stimulus to induce eye movement of a user, sensing the eye movement of the user in response to the visual stimulus, and displaying a result as to whether the user has successfully accomplished a mission in response to the visual stimulus according to a result of the sensing.

### CROSS-REFERENCE TO RELATED APPLICATION

This application claims the benefit of Korean Patent Application No. 10-2014-0120849 filed on Sep. 12, 2014, the disclosures of which are incorporated herein by reference.

### TECHNICAL FIELD

The embodiments described herein pertain generally to a method and an apparatus for providing a prefrontal activity game.

### BACKGROUND

The attention deficit and hyperactivity disorder (hereinafter, “ADHD”) is a common mental disorder that appears in early childhood, and the main symptoms of ADHD include inattention, hyperactivity and impulsivity. Since individuals with attention deficit and hyperactivity have difficulties in their daily lives, like learning, continuing to work, completing tasks and so on, they are prone to be socially maladjusted.

Factors causing ADHD include genetic factors, biochemical factors, which are associated with a lead level and a reaction to food additives of instant food, neurobiological factors, which are associated with a problem in delivery of a stimulus to the brain, circumferential environmental factors, and others. Among the factors, neuroscientific factors are regarded as critical, and thus, researches on medicines for treating ADHD and the neuroscientific factors have been actively performed.

Abnormality of the prefrontal cortex in a neuroanatomical aspect, abnormality of neurotransmitters of the central nervous system in a neurobiochemical aspect, and abnormality of the functions of the prefrontal cortex, the thalamus, and the dopaminergic mesocortical projection in a neurobiological aspect have been researched. However, the exact causes for ADHD have been unknown, and people estimate that ADHD results from the complex operation of the above-described causes (H. Mang, H. Chung, The Journal of Elementary Education, 2005).

Drug most commonly used for treatment of ADHD is a central nerve stimulant, and Ritaline, Methylphen and others containing methylphenidate as a component are used.

In this regard, Korean Patent Application Publication No. 10-2013-0041012 (Title of Invention: ADHD Model Mouse, Method for Verifying Effect of Preventing and Reducing Attention Deficit Disorder by Using the Model Mouse, and Composition for Preventing and Treating Attention Deficit Disorder, which includes Non-Specific T-Type Calcium Inhibitor) describes using an ADHD model mouse for attention deficit disorder medicine screening based on results obtained from observation of brainwaves with low frequencies, hyperactivity, and deterioration of the cognitive function, which are the symptoms of the attention deficit disorder.

However, while such drug is known to have a short-term effect in reducing inattention, hyperactivity and impulsivity, it has problems in that it has not been verified with regard to whether to have a long-term effect and may cause side effects.

Meanwhile, as a method for activating the prefrontal without using drug, a brain development system by simple visual suggestion or brainwave inducement has been developed, but there are few devices capable of identifying the state of a user in real time, and there are many problems in view of reliability of a treatment effect.

### SUMMARY

Some of example embodiments provide a method for providing a prefrontal activity game to activate the prefrontal without using drug.

Also, some of example embodiments provide a system, which can operate in a mobile system, and thus, identify the state of a user in real time.

However, the problems sought to be solved by the present disclosure are not limited to the above description, and other problems can be clearly understood by those skilled in the art from the following description.

In accordance with a first aspect of example embodiments, a method for providing a prefrontal activity game is provided. The method includes providing a visual stimulus to induce eye movement of a user, sensing the eye movement of the user in response to the visual stimulus, and displaying a result as to whether the user has successfully accomplished a mission in response to the visual stimulus according to a result of the sensing.

In accordance with a second aspect of example embodiments, an apparatus for providing a prefrontal activity game is provided. The apparatus includes a display; a memory comprising an application for prefrontal activity; and a processor executing the application. The processor provides the display with a visual stimulus to induce eye movement of a user according to execution of the application, senses the eye movement of the user in response to the visual stimulus, and displays a result as to whether the user has successfully accomplished a mission in response to the visual stimulus according to a result of the sensing.

In accordance with example embodiments, the method for providing a prefrontal activity game senses movement of the eyes of a user by a visual stimulus to provide compensation according to a result of the sensing. Accordingly, the user receives the compensation, and as a result, an effect in activating the prefrontal is achieved.

Furthermore, there are significant effects in that it is possible to improve attention and play an online game with interest and entertainment through the prefrontal activity game.

If the technology suggested by example embodiments is used, natural articulation of a patient with abnormality in his/her prefrontal is induced, so that there is no side effect resulting from drug.

In addition, if the technology suggested by example embodiments operates in a mobile device, it is possible to identify the state of a user in real time.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

In the detailed description that follows, embodiments are described as illustrations only since various changes and modifications will become apparent to those skilled in the art from the following detailed description. The use of the same reference numbers in different figures indicates similar or identical items.

FIG. 1 is a conceptual view of a system for providing a prefrontal activity game in accordance with an example embodiment.

FIG. 2 is a block diagram of a prefrontal activity apparatus for activating the prefrontal.

FIG. 3 is a flow chart showing a method for providing a prefrontal activity game in accordance with an example embodiment.

FIG. 4A shows an interface screen for a prefrontal activity game in accordance with an example embodiment, and FIG. 4B shows a feedback bar displayed in the interface of the prefrontal activity game in accordance with an example embodiment.

FIG. 5A and FIG. 5B show activity of the prefrontal when the prefrontal activity apparatus in accordance with an example embodiment is used.

### DETAILED DESCRIPTION

Hereinafter, example embodiments will be described in detail with reference to the accompanying drawings so that inventive concept may be readily implemented by those skilled in the art. However, it is to be noted that the present disclosure is not limited to the example embodiments but can be realized in various other ways. In the drawings, certain parts not directly relevant to the description are omitted to enhance the clarity of the drawings, and like reference numerals denote like parts throughout the whole document.

Throughout the whole document, the terms “connected to” or “coupled to” are used to designate a connection or coupling of one element to another element and include both a case where an element is “directly connected or coupled to” another element and a case where an element is “electronically connected or coupled to” another element via still another element.

Further, the term “comprises or includes” and/or “comprising or including” used in the document means that one or more other components, steps, operations, and/or the existence or addition of elements are not excluded in addition to the described components, steps, operations and/or elements.

Hereinafter, the method for providing a prefrontal activity game in accordance with an example embodiment is described in detail with reference to the accompanying drawings.

Here, the prefrontal activity game has a specific object in activating the prefrontal. Thus, the system for providing the prefrontal activity game is first described.

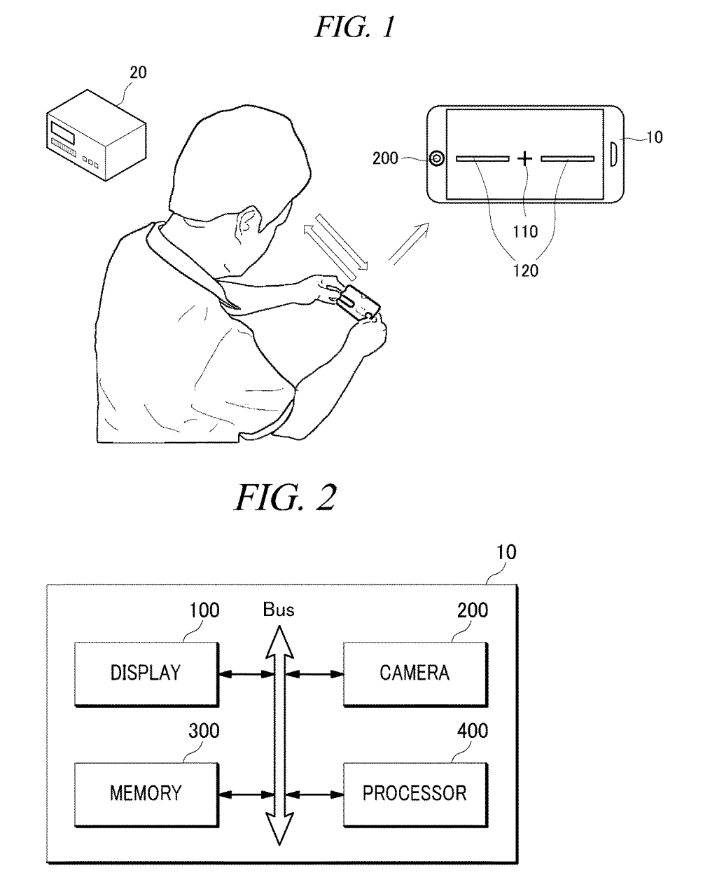

FIG. 1 is a conceptual view of the system for providing the prefrontal activity game in accordance with an example embodiment.

With reference to FIG. 1, the system for providing the prefrontal activity game includes a prefrontal activity apparatus 10, which provides the prefrontal activity game.

The prefrontal activity apparatus 10 may provide a visual stimulus to induce movement of the eyes of a user. In addition, since the prefrontal activity apparatus 10 has a camera module, it can sense the eye movement of the user by the visual stimulus. According to a result of the sensing, the prefrontal activity apparatus 10 may display a result with regard to whether the user has successfully accomplished a mission in response to the visual stimulus.

For reference, the prefrontal activity apparatus 10 may also be realized in a mobile device assuring portability and mobility. Such a radio communication device may include any type of a handheld-based wireless communication device such as a personal communication system (PCS), a global system for mobile communication (GSM), a personal digital cellular (PDC), a personal handyphone system (PHS), a personal digital assistant (PDA), international mobile telecommunication (IMT)-2000, code division multiple access (CDMA)-2000, W-code division multiple access (W-CDMA), a wireless broadband Internet (Wibro) terminal, a smart phone, a smart pad, a tablet PC and so on. Especially, the prefrontal activity apparatus 10 is an intelligent device developed by adding computer supporting functions such as Internet communication and information search to a mobile device, and may be a smart phone capable of downloading and executing user desired multiple application programs (i.e., applications). In addition, the prefrontal activity apparatus 10 may be realized in a form of a device dedicated to provide the prefrontal activity game, as well as a widely used computer such as a desktop and a laptop.

The prefrontal activity system may further include external measurement equipment 20. In addition to the method that tracks the eye movement by means of the camera module, it is possible to more accurately track the eye movement, by using the external measurement equipment 20, which measures electro-oculogram. For reference, the electro-oculogram is a potential recorded by an electrode attached to the skin around the eyes, and can detect the eye movement. In general, since the eyeball always has a potential difference like a positive charge in the cornea and a negative charge of the posterior pole, irrespective of a response to an external stimulus, an electrode may be attached to the area surrounding the eyes so as to cause change in an electric field, accompanied by the eye movement. That is, the external measurement equipment 20 can track the eye movement from an EOG signal collected from a sensor attached to the periphery of the eyes of the user.

In addition, information of the tracked eye movement may be delivered to the prefrontal activity apparatus 10, to be used to display a result with regard to whether the user has successfully accomplished a mission in response to the visual stimulus. Accordingly, the information may be utilized in the normal operation of the prefrontal activity apparatus 10.

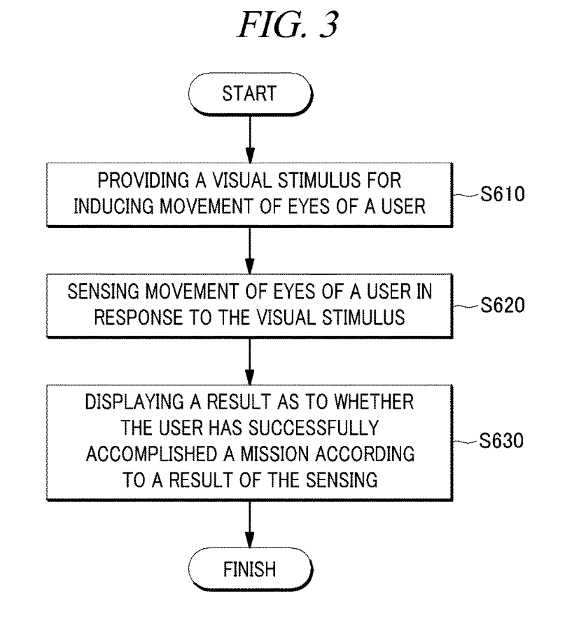

Next, the prefrontal activity apparatus 10 is described in more detail with reference to FIG. 2.

FIG. 2 is a block diagram of the prefrontal activity apparatus for activating the prefrontal.

The prefrontal activity apparatus 10 may include a display 100, a camera 200, a memory 300 including an application program for prefrontal activity, and a processor 400.

The prefrontal activity apparatus 10 displays an application to be executed in response to the eye movement of the user in the display 100.

The display 100 may display a mark unit 110, a feedback bar 120 and others according to the execution of the application for prefrontal activity.

In addition, the processor 500 may control the display 100 to display the mark unit 110, the feedback bar 102 and others according to the execution of the application for prefrontal activity.

In addition, the prefrontal activity apparatus 10 may display use stages, time, and the operation state in the display 100. Here, the use stages may be divided into “standby,” “stimulation,” “compensation” and others.

In addition, while the application for prefrontal activity is executed with a start signal, the “standby,” “stimulation,” and “compensation” stages may be implemented for certain time. The display 100 may display lapse of the time according to convenience of the user. In addition, the certain time may be controlled to be appropriate for the user in the application for prefrontal activity.

In addition, the display 100 may display the operation state in accordance with each of the stages. That is, in the “standby” state, the mark unit 110 may display a first mark for drawing attention, and in the “stimulation” state, the mark unit 110 may display a left or right arrow to guide the eye movement of the user. In this case, the feedback bar 120 may display position information of the eyes of the user in real time. In addition, in the “compensation” state, the mark unit 110 may display “success” or “fail” to display a result with regard to whether the user has successfully accomplished a mission.

In other words, the mark unit 110 may display a visual stimulus. In addition, the user can control movement of his/her eyes in accordance with the visual stimulus displayed in the mark unit 110. In addition, the mark unit 110 may display a result for the movement of the eyes of the user.

The feedback bar 120 may display the state of the eye movement of the user in response to the arrow. In addition, the feedback bar 120 may display a degree of the eye movement state in proportion to a degree of the eye movement of the user from the position indicated by the first mark.

That is, the feedback bar 120 may display the position information of the eyes according to images of the eye movement of the user photographed by the camera 200. In this case, the feedback bar 120 may display the result of the eye movement in different shapes or colors. In addition, the images photographed by the camera 200 may be stored in the memory 300. The stored images may be calculated as current position information of the user's eyes in the application for prefrontal activity to be displayed in the feedback bar 120.

In addition, the prefrontal activity apparatus 10 photographs the eye movement of the user through the camera 200. In case of a smart device, an already equipped camera module may be used, and in case of a widely used computer, an already equipped module or a separate outer mounted camera module may be used. In addition, in case of a device dedicated to provide the prefrontal activity game, the camera 200 is positioned in the central area of the device along one side surface thereof, so as to more accurately sense the eye movement of the user.

Returning to FIG. 2, the memory 300 of the prefrontal activity apparatus 10 may include the application for prefrontal activity. The application for prefrontal activity may have a function to photograph and store the eye movement of the user in real time through the camera module of the prefrontal activity apparatus 10, a function to analyze the photographed eye movement of the user and determine information of a position indicated by the eye movement, and other functions.

In addition, the memory 300 may store an operation system (OS) for the operation of the prefrontal activity apparatus 10, various applications, and others. In addition, the memory 300 may include a storage in a volatile or non-volatile memory form, and the storage may store information of photographs obtained by photographing the eye movement of the user. Since the configuration technology of the memory 300 is conventionally well-known, the detailed descriptions thereof are omitted hereinafter.

Returning to FIG. 2, the processor 400 provides the display 100 with a visual stimulus for inducing the eye movement of the user according to the execution of the application for prefrontal activity, senses the eye movement of the user in response to the visual stimulus, and displays a result with regard to whether the user has successfully accomplished a mission in response to the visual stimulus, according to the sensing results.

In addition, the processor 400 may display the feedback bar 120 showing the state of the eye movement of the user in accordance with the arrow.

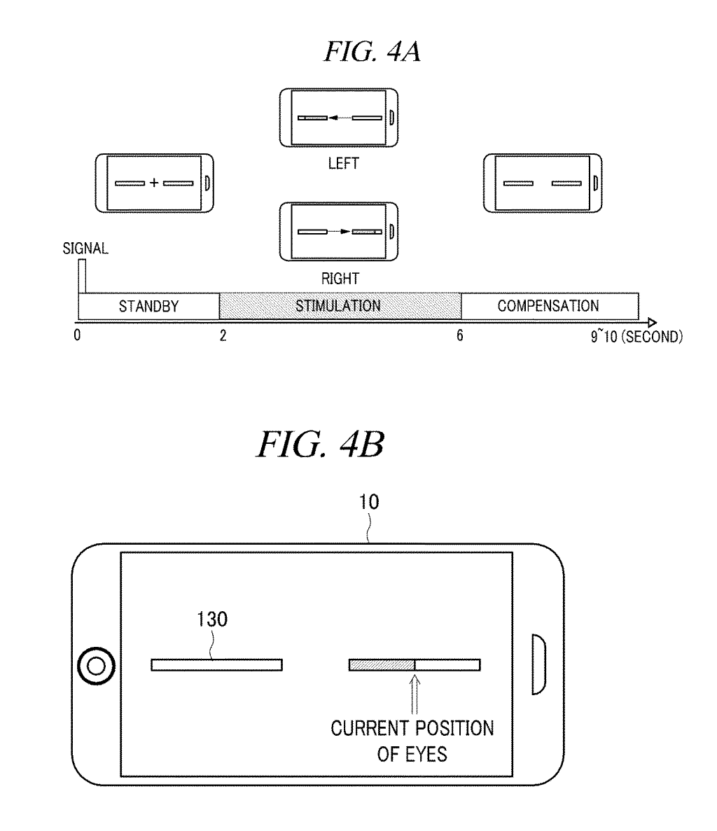

Hereinafter, the method for providing the prefrontal activity game in accordance with an example embodiment is described in detail with reference to FIG. 3 to FIG. 5.

First, FIG. 3 is a flow chart showing the method for providing the prefrontal activity game in accordance with an example embodiment.

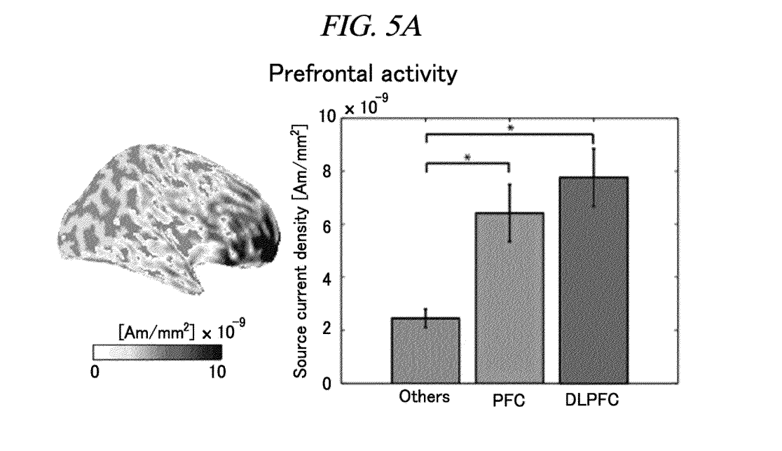

In addition, FIG. 4A shows an interface screen of the prefrontal activity game in accordance with an example embodiment, and FIG. 4B shows a feedback bar displayed in the interface of the prefrontal activity game in accordance with an example embodiment.

With reference to FIG. 3, the method for providing the prefrontal activity game includes providing a visual stimulus for inducing the eye movement of the user (S610), sensing the eye movement of the user in response to the visual stimulus (S620), and displaying a result with regard to whether the user has successfully accomplished a mission in response to the visual stimulus, according to the result of the sensing (S630).

First, the prefrontal activity apparatus 10 provides a visual stimulus for inducing the eye movement of the user (S610). In this case, the prefrontal activity apparatus 10 may display the visual stimulus on the display 100.

Here, the visual stimulus may be displayed on the mark unit 110. The mark unit 110 is positioned in the center of the prefrontal activity apparatus 10, and may display the first mark, left and right arrows, and others.

That is, the prefrontal activity apparatus 10 may display the first mark, so as to guide the user to look at the first mark for certain time. In addition, in the “standby” state, the display 100 may display the mark unit 110.

In this case, the first mark is positioned in the center of the display 100 or the game interface displayed on the display 100, and may be realized in various shapes or colors. For example, a mark in a cross or heart shape may be used, but the present disclosure is not limited thereto. Marks in various shapes may be used depending on user's selection.

Here, with reference to FIG. 4A, the prefrontal activity apparatus 10 may identify that the first mark in a cross shape is displayed in the mark unit 110. In this case, the user can look at the first mark, and the period of that moment may be referred to as a “standby” period.

In addition, the prefrontal activity apparatus 10 may display a left or right arrow after lapse of the certain time, so as to guide the user to look at the position indicated by the corresponding arrow for certain time.

In this case, the arrow is positioned in the center of the prefrontal activity apparatus 10, and may be used as the visual stimulus for inducing the eye movement of the user. The arrow may be formed in various colors or shapes.

Returning to FIG. 4A, the period of that moment is referred to as a “stimulation” period, and the prefrontal activity apparatus 10 may display a right or left arrow. In this case, the user can look at the direction indicated by the arrow.

Next step (S620), the prefrontal activity apparatus 10 senses the eye movement of the user in response to the visual stimulus in the previous stage (S610).

That is, the prefrontal activity apparatus 10 may track the eye movement based on images of the user inputted by the camera provided in the prefrontal activity apparatus, which executes the prefrontal activity game.

In other words, the prefrontal activity apparatus 100 may sense the eye movement of the user through the camera module for certain time. The application for prefrontal activity may process real-time information of a current position of the eyes based on the captured image information as describe above. Accordingly, the position, at which the eyes of the user look, may be displayed in the feedback bar 120 in real time.

In this case, with reference to FIG. 4B, the feedback bar 120 is positioned at each of left and right sides of the mark unit 110, and may be provided in a bar graph form extending toward a horizontal direction. The feedback bar 120 may express the current position, at which the eyes of the user look, by filling the inside of the feedback bar 120 with a different color according to the eye movement of the user.

Accordingly, since the user can identify the current position, at which his/her eyes moving due to the visual stimulus look, from the feedback bar 120, he/she can receive a feedback in response to his/her behavior. As a result, the user's attention can be improved.

Next step (S630), the prefrontal activity apparatus 100 displays a result with regard to whether the user has successfully accomplished a mission in response to the visual stimulus, according to the result of the sensing in the previous step (S620).

In this case, the prefrontal activity apparatus 100 may display “success” or “fail” in the mark unit 110 with regard to whether the user has successfully accomplished a mission.

Meanwhile, the prefrontal activity apparatus 10 may sense the eye movement of the user, but the external measurement equipment 20 may sense the eye movement of the user.

In accordance with another example embodiment, in step S620, the external measurement equipment 20 tracks the eye movement from an EOG signal collected from the sensor attached to the periphery of the eyes of the user.

For example, the EOG signal may be measured through two silver chloride (Ag/AgCl) electrodes attached onto the forehead of the user. EOG is a cornea-retina potential signal generated by eyeball movement. In general, for EOG, a horizontal signal for left and right movement of the eyes can be obtained from both ends of the forehead, and a vertical signal for upward and downward movement of the eyes can be obtained from the upper and lower parts of the eyes. EOG may be measured in the external measurement equipment 20.

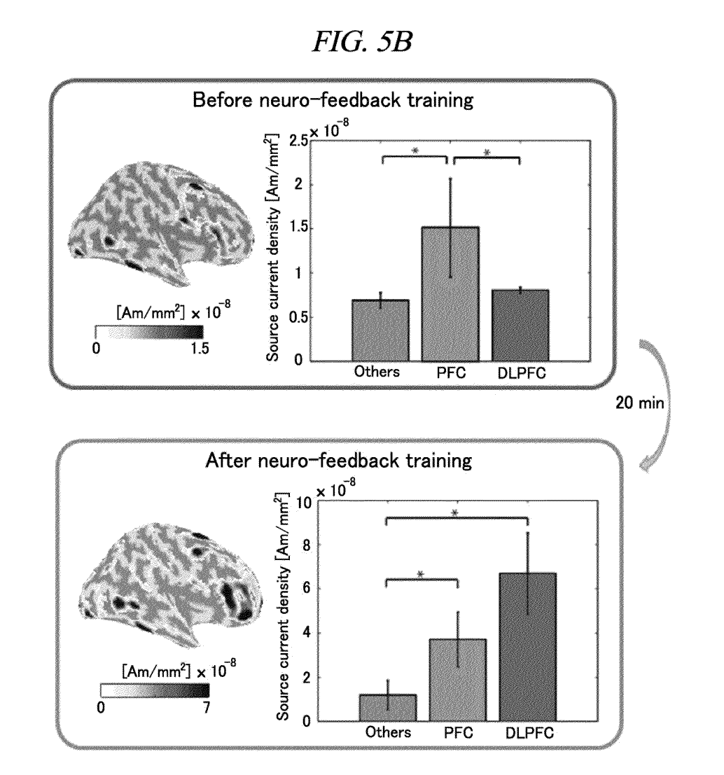

Next, FIG. 5A and FIG. 5B show prefrontal activity when the prefrontal activity apparatus in accordance with an example embodiment is used.

FIG. 5A shows a photograph and a graph showing comparison of the activity of the prefrontal with other areas when the prefrontal activity apparatus 10 is used. The right graph compares the prefrontal cortex (PFC), the dorsolateral prefrontal cortex (DLPFC), and other areas together. The result values in the graph show average values of the respective areas. Especially, the dorsolateral prefrontal cortex (DLPFC) is known to be essential for the cognitive function. In accordance with an example embodiment, the user implemented a task by using the prefrontal activity apparatus 10 of example embodiments for about ten minutes, and as a result, it was identified that the prefrontal activity was three times the other areas.

Next, FIG. 5B shows the prefrontal activity when an eight year-old ADHD patient uses the prefrontal activity apparatus.

Meanwhile, ADHD stands for an attention deficit and hyperactivity disorder. ADHD commonly develops in childhood, and have symptoms of distraction, hyperactivity, and impulsivity due to continuous deficit of attention. If kids with the symptoms are left untreated, they may continuously suffer from difficulties in their childhood lives, and some of them may still have the symptoms in teenagerhood and adulthood.

FIG. 5B shows a result obtained when an eight-year old ADHD patient uses the prefrontal activity apparatus 10. It is identified that while the prefrontal is activated in the initial stage of the test as shown in the left drawing of FIG. 5B, the dorsolateral prefrontal cortex (DLPFC) essential for the cognitive function is not activated. However, it is identified that as a result of implementing the task for twenty minutes, the dorsolateral prefrontal cortex (DLPFC) has been strongly activated (increase of about 6 times). As aforementioned, the dorsolateral prefrontal cortex (DLPFC) performs a critical role for the cognitive function.

The system for providing the prefrontal activity game in accordance with an example embodiment may be embodied in a storage medium including instruction codes executable by a computer or processor such as a program module executed by the computer or processor. A computer readable medium can be any usable medium which can be accessed by the computer and includes all volatile/nonvolatile and removable/non-removable media. Further, the computer readable medium may include all computer storage and communication media. The computer storage medium includes all volatile/nonvolatile and removable/non-removable media embodied by a certain method or technology for storing information such as computer readable instruction code, a data structure, a program module or other data. The communication medium typically includes the computer readable instruction code, the data structure, the program module, or other data of a modulated data signal such as a carrier wave, or other transmission mechanism, and includes information transmission mediums.

The above description of the example embodiments is provided for the purpose of illustration, and it would be understood by those skilled in the art that various changes and modifications may be made without changing technical conception and essential features of the example embodiments. Thus, it is clear that the above-described example embodiments are illustrative in all aspects and do not limit the present disclosure. For example, each component described to be of a single type can be implemented in a distributed manner. Likewise, components described to be distributed can be implemented in a combined manner.

The scope of the inventive concept is defined by the following claims and their equivalents rather than by the detailed description of the example embodiments. It shall be understood that all modifications and embodiments conceived from the meaning and scope of the claims and their equivalents are included in the scope of the inventive concept.

We claim:

1. A method for providing a prefrontal activity game, comprising:
providing a visual stimulus to induce eye movement of a user;
sensing the eye movement of the user in response to the visual stimulus; and
displaying a result as to whether the user has successfully accomplished a mission in response to the visual stimulus according to a result of the sensing.

2. The method for providing a prefrontal activity game of claim 1,
wherein the providing of the visual stimulus comprises:
displaying a first mark to guide the user to look at the first mark for predetermined time; and
displaying a left or right arrow after lapse of the predetermined time, to guide the user to look at a position indicated by the corresponding arrow for predetermined time.

3. The method for providing a prefrontal activity game of claim 2, further comprising
displaying a feedback bar showing the state of the eye movement of the user in response to the arrow,
wherein the feedback bar displays a degree of the state of the eye movement in proportion to a degree of the eye movement of the user from the position indicated by the first mark.

4. The method for providing a prefrontal activity game of claim 1,
wherein the sensing of the eye movement tracks the eye movement based on images of the user taken by a camera equipped in the prefrontal activity apparatus, which executes the prefrontal activity game.

5. The method for providing a prefrontal activity game of claim 1,
wherein the sensing of the eye movement tracks the eye movement based on an EOG signal collected from a sensor attached to the periphery of the eyes of the user.

6. A computer readable recording medium, which records a program for implementing the method of claim 1 in a computer.

7. An apparatus for providing a prefrontal activity game, comprising:
a display;
a memory comprising an application for prefrontal activity; and
a processor executing the application,
wherein the processor provides the display with a visual stimulus to induce eye movement of a user according to execution of the application,
senses the eye movement of the user in response to the visual stimulus, and
displays a result as to whether the user has successfully accomplished a mission in response to the visual stimulus according to a result of the sensing.

8. The apparatus for providing a prefrontal activity game of claim 7,
wherein the processor displays a first mark to guide the user to look at the first mark for predetermined time, and displays a left or right arrow after lapse of the predetermined time, to guide the user to look at the position indicated by the corresponding arrow for predetermined time.

9. The apparatus for providing a prefrontal activity game of claim 8,
wherein the processor displays a feedback bar showing the state of the eye movement of the user in response to the arrow, and the feedback bar displays a degree of the state of the eye movement in proportion to a degree of the eye movement of the user from the position indicated by the first mark.

10. The apparatus for providing a prefrontal activity game of claim 7,
wherein the processor tracks the eye movement based on images of the user taken by a camera provided in the prefrontal activity apparatus, which executes the prefrontal activity game.

11. The apparatus for providing a prefrontal activity game of claim 7,
wherein the processor tracks the eye movement based on an EOG signal collected from a sensor attached to the periphery of the eyes.
